(19) United States
(12) Patent Application Publication
(10) Pub. No.: US 2026/0335216 A1
(43) Pub. Date: Oct. 29, 2026

(54) COMPOSITION COMPRISING HIGH MOLECULAR WEIGHT HYALURONIC ACID MICROPARTICLES

(71) Applicant: AMOREPACIFIC CORPORATION, Seoul (KR)

(72) Inventors: Hyoseok AN, Yongin-si, Gyeonggi-do (KR); Chaeyeon SONG, Yongin-si, Gyeonggi-do (KR); Eun Soo LEE, Yongin-si, Gyeonggi-do (KR); Seunghan PARK, Yongin-si, Gyeonggi-do (KR); Somi LEE, Yongin-si, Gyeonggi-do (KR); Joonho CHOI, Yongin-si, Gyeonggi-do (KR)

(73) Assignee: AMOREPACIFIC CORPORATION, Seoul (KR)

(21) Appl. No.: 19/110,483

(22) PCT Filed: Sep. 13, 2023
(86) PCT No.: PCT/KR2023/013702

§ 371 Date: Mar. 11, 2025

(30) Foreign Application Priority Data
Sep. 15, 2022 (KR) 10-2022-0116111
Sep. 15, 2022 (KR) 10-2022-0116133

(51) Int. Cl. A61K 8/73; A61K 8/362; A61K 8/67; A61K 9/00; A61K 31/375; A61K 47/02; A61K 47/10; A61K 47/12; A61K 47/18; A61K 47/22; A61K 47/26; A61K 47/36; A61Q 19/00; A61Q 19/02; A61Q 19/08

(52) U.S. Cl. CPC A61K 8/735; A61K 8/362; A61K 8/676; A61K 9/0014; A61K 31/375; A61K 47/02; A61K 47/10; A61K 47/12; A61K 47/183; A61K 47/22; A61K 47/26; A61K 47/36; A61Q 19/007; A61Q 19/02; A61Q 19/08; A61K 2800/413

(57) ABSTRACT

A composition comprising high molecular weight hyaluronic acid microparticles is disclosed in the present specification. In the present disclosure, high molecular weight hyaluronic acids are changed into microparticles so that high molecular weight hyaluronic acids can easily penetrate the skin even without having a low molecular weight and can be stably included in a formulation. In addition, after penetrating the skin and being neutralized to match the pH of the skin, particulate high molecular weight hyaluronic acids included in the composition of the present disclosure expand again in size, and thus can provide excellent moisturization and elasticity improvement effects to the skin.

### CROSS-REFERENCE TO RELATED APPLICATIONS

This application is a 35 U.S.C. § 371 National Stage Entry of International Patent Application No. PCT/KR2023/013702, filed Sep. 13, 2023, claiming benefit from Korean Patent Application No. 10-2022-0116111, filed Sep. 15, 2022, and Korean Patent Application No. 10-2022-0116133, filed Sep. 15, 2022, the entire contents of which are hereby incorporated by this reference and priority is claimed to each of the foregoing.

### TECHNICAL FIELD

The present specification discloses a composition comprising high molecular weight hyaluronic acid microparticles.

### BACKGROUND

Hyaluronic acid (HA) is a straight-chain polysaccharide composed of alternately linked β-acetylglucosamine and β-glucuronic acid, which is distributed in the human body in connective tissues such as subcutaneous tissue and cartilage tissue. The hyaluronic acid has excellent biocompatibility and is known to have beneficial effects on the skin, including increasing skin's moisture retention, maintaining skin elasticity, and improving skin barrier function. However, natural hyaluronic acid is not structurally stable and is easily broken down and eliminated in the human body, thus research has been conducted to stabilize natural hyaluronic acid.

A molecular weight of the hyaluronic acid varies depending on a length of a sugar chain. The hyaluronic acid with a low molecular weight is easy to penetrate the skin, but it has a short duration of stay in the skin and a limited ability to retain water, which leads to problems of less effectiveness on the skin, changes in odor, discoloration, and the like. The hyaluronic acid with a high molecular weight has excellent moisturizing and regenerating properties for the skin, but is difficult to penetrate the skin. Therefore, there has been a limitation in realizing the effect of hyaluronic acid on the skin when applied to actual formulations unless the hyaluronic acid is administered directly into the skin through injections.

In addition, in the cosmetics or pharmaceutical industry, when an active substance that is difficult to penetrate the skin or has low stability is included in a skin external application formulation, methods of promoting the absorption of the active substance into the skin using synthetic surfactants, etc. or increasing the permeability of the active substance into the skin using synthetic polymer compounds such as polyethylene glycol or liposome nanoparticles as a delivery agent have been widely used. However, issues such as the human toxicity and allergenicity of amphiphilic enhancers used in synthetic compounds or liposome nanoparticles, as well as their harmful effects on the environment, have become concerns. As a result, the cosmetics and pharmaceutical industries have recently been striving to avoid their use. Therefore, there is a need to develop delivery agents that are both human- and environmentally friendly while effectively enhancing the transdermal absorption of active substances.

### SUMMARY OF THE INVENTION

An object to be achieved by the present disclosure is to provide a composition and method of manufacturing the same comprising a high molecular weight hyaluronic acid that facilitates skin penetration without depolymerization or cross-linking.

An object to be achieved by the present disclosure is to provide a composition and method of manufacturing the same comprising a high molecular weight hyaluronic acid that is stable in a formulation.

An object to be achieved by the present disclosure is to provide a carrier composition for transdermal delivery of an active substance that is biocompatible and facilitates skin penetration.

An object to be achieved by the present disclosure is to provide a skin external application composition comprising an active substance and a carrier composition for the transdermal delivery of the active substance.

To achieve the above-mentioned objects, there is provided a composition, according to an embodiment of the present disclosure, comprising: a high molecular weight hyaluronic acid or salt particles thereof; and an acidic pH adjuster, in which the acidic pH adjuster is an organic acid or inorganic acid, and in which the high molecular weight hyaluronic acid or salt particles thereof have an average particle diameter of 100 nm or less, and wherein a pH of the composition is 3.0 or more but less than 5.0.

In addition, there is provided a method of manufacturing the composition, according to an embodiment of the present disclosure, comprising: micronizing a high molecular weight hyaluronic acid or salt thereof by adding an acidic pH adjuster to a solution that comprises the high molecular weight hyaluronic acid or salt thereof, in which the micronized high molecular weight hyaluronic acid or salt particle thereof has an average particle diameter of 100 nm or less, and a pH of the composition is 3.0 or more but less than 5.0.

In addition, there is provided a carrier composition for transdermal delivery of an active substance, according to an embodiment of the present disclosure, comprising: a high molecular weight hyaluronic acid or salt particle thereof; and an acidic pH adjuster, in which the acidic pH adjuster is an organic acid or inorganic acid, and in which the high molecular weight hyaluronic acid or salt particle thereof has an average particle diameter of 100 nm or less, wherein a pH of the composition is 3.0 or more but less than 5.0, and wherein the composition releases the active substance at a pH of 5 to 7.

In addition, there is provided a skin external application composition, according to an embodiment of the present disclosure, comprising: an active substance; and a carrier for transdermal delivery of the active substance, comprising the aforementioned composition.

An embodiment of the present disclosure may provide a composition comprising a high molecular weight hyaluronic acid microparticle that facilitates skin penetration without depolymerization by micronizing the high molecular weight hyaluronic acid and that is stably comprised in a formulation. The micronized high molecular weight hyaluronic acid according to an embodiment of the present disclosure penetrates the skin and then is neutralized to pH of the skin and expanded in size again, which can provide excellent benefits for moisturizing the skin and improving skin elasticity. In addition, according to an embodiment, the skin external application composition comprising an active substance and a carrier for the transdermal delivery of the active substance, comprising the aforementioned composition, can exhibit dual functionality in that the skin external application composition penetrates the skin, releases the active substance, and then the carrier itself also can provide beneficial effects to the skin.

### BRIEF DESCRIPTION OF THE DRAWINGS

FIG. 1 is a graph comparing a particle size of a composition (Example 1) according to an embodiment of the present disclosure with that of Comparative Example 1.

FIG. 2 is a graph comparing particle sizes of compositions (Examples 1 to 4) according to an embodiment of the present disclosure with that of Comparative Example 2.

FIG. 3 is a graph illustrating molecular weights of a high molecular weight hyaluronic acid microparticle included in a composition of Comparative Example 1.

FIG. 4 is a graph illustrating molecular weights of a high molecular weight hyaluronic acid microparticle included in the composition (Example 1) according to an embodiment of the present disclosure.

FIG. 5 is a view confirming depths of absorption into skin tissue of the composition (Example 1) according to an embodiment of the present disclosure and that of Comparative Example 1.

FIG. 6 illustrates a schematic view of the active substance delivery and release system of a carrier composition according to an embodiment of the present disclosure.

FIG. 7 is a view confirming the depth of active substance absorption in skin tissue according to Example 5 according to an embodiment of the present disclosure and Comparative Example 3.

### DETAILED DESCRIPTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to include all modifications, equivalents, or substitutions that fall within the scope of the spirit and art of the present disclosure. Singular expressions include plural expressions unless clearly described as different meanings in the context. In the present application, the terms “comprises,” “comprising,” “includes,” “including,” “containing,” “has,” “having” or other variations thereof are inclusive and therefore specify the presence of stated features, integers, steps, operations, elements, components, and/or combinations thereof, but do not exclude the presence or addition of one or more other features, integers, steps, operations, elements, components, and/or combinations thereof.

An embodiment of the present disclosure may provide a composition comprising: a high molecular weight hyaluronic acid or salt particle thereof; and an acidic pH adjuster, in which the acidic pH adjuster is an organic acid or inorganic acid, and in which the high molecular weight hyaluronic acid or salt particle thereof has an average particle diameter of 100 nm or less, and wherein a pH of the composition is 3.0 or more but less than 5.0.

The term “hyaluronic acid” in the present specification refers to an anionic natural polysaccharide that exists in salt form and is a raw material corresponding to EWG rating 1. The hyaluronic acid is mainly produced and purified by microorganisms and is present in the connective tissues and extracellular matrix of the human body, and is often used as a moisturizer because of a high moisture content due to anionic functional groups. A molecular structure of the hyaluronic acid may be represented by Chemical Formula 1 below, and may be comprised in the present specification in the form of a salt of the hyaluronic acid.

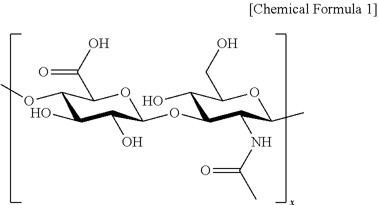

where x is 200 to 20000 mol.

In an embodiment of the present disclosure, the salt may not be specifically limited, but may be acid addition salt, base addition salt, or amino acid salt. For example, the salt may be inorganic acid salts such as hydrochloride, hydrobromide, sulfate, hydrogen iodide, nitrate, phosphate, etc., organic acid salts, such as citrate, oxalate, acetate, formate, propionate, benzoate, trifluoroacetate, maleate, tartrate, methanesulfonate, benzenesulfonate, paratoluenesulfonate, etc., inorganic base salt, such as sodium salt, potassium salt, calcium salt, magnesium salt, copper salt, zinc salt, aluminum salt, ammonium salt, etc., organic base salt, such as triethylammonium salt, triethanolammonium salt, pyridinium salt, diisopropylammonium salt, etc., or amino acid salt, such as lysine salt, arginine salt, histidine salt, aspartate, glutamate, etc. In an embodiment, the salt may be sodium hyaluronate, potassium hyaluronate, hyaluronic acid acetate, hyaluronic acid ammonium salt, or C12-13 alkyl glyceryl hydrolyzed hyaluronate. In the present disclosure, the high molecular weight hyaluronic acid or salt thereof has a weight average molecular weight that varies with a length of a sugar chain, and in an embodiment, the weight average molecular weight of the high molecular weight hyaluronic acid or salt thereof may be 5 to 10,000 kDa. Specifically, the high molecular weight hyaluronic acid or salt thereof may have a weight average molecular weight of 5 kDa or more, 10 kDa or more, 100 kDa or more, 500 kDa or more, 1000 kDa or more, 1,100 kDa or more, 1,200 kDa or more, 1,300 kDa or more, 1,400 kDa or more, 1,500 kDa or more, 1,600 kDa or more, 1,700 kDa or more, 1,800 kDa or more, 1,900 kDa or more, 2,000 kDa or more, 2,200 kDa or more, 2,400 kDa or more, 2,600 kDa or more, 2,800 kDa or more, 2,900 kDa or more, 3,000 kDa or more, 4,000 kDa or more, or 5,000 kDa or more, and may have a weight average molecular weight of 10,000 kDa or less, 9,000 kDa or less, 8,000 kDa or less, 7,000 kDa or less, 6,000 kDa or less, 5,000 kDa or less, 4,500 kDa or less, 4,000 kDa or less, 3,900 kDa or less, 3,800 kDa or less, 3,700 kDa or less, 3,600 kDa or less, 3,500 kDa or less, 3,400 kDa or less, 3,300 kDa or less, 3,200 kDa or less, 3,100 kDa or less, 3,000 kDa or less, 2,500 kDa or less, 2,000 kDa or less, 1,900 kDa or less, 1,800 kDa or less, 1,700 kDa or less, 1,600 kDa or less, 1,500 kDa or less, 1,000 kDa or less, 500 kDa or less, 100 kDa or less, or 10 kDa. More specifically, the weight average molecular weight of the high molecular weight hyaluronic acid or salt thereof may be 1,000 to 5,000 kDa. When the weight average molecular weight of the high molecular weight hyaluronic acid or salt thereof is less than 5 kDa, the moisturizing effect may be reduced, and when the weight average molecular weight is more than 10,000 kDa, the high molecular weight hyaluronic acid or salt thereof may have excessively high viscosity or may have limited micronization, which makes it difficult to penetrate the skin.

In an embodiment of the present disclosure, as high molecular weight hyaluronic acid particles are comprised with the acidic pH adjuster, a net charge in the molecule of negatively charged hyaluronic acid is adjusted by hydrogen cations generated by acid from the acidic pH adjuster while the molecular weight of the high molecular weight hyaluronic acid is maintained. Therefore, the high molecular weight hyaluronic acid particles may be micronized into nano-sized particles. In more detail, as the hydrogen cations of the acidic pH adjuster increase in a solution and neutralize the negative charge of the high molecular weight hyaluronic acid, the charge density of the high molecular weight hyaluronic acid decreases. Therefore, polymer chains of the high molecular weight hyaluronic acid may have a spherical shape instead of being linear in the solution. That is, since radius of gyration (globular size) of the polymer chain is reduced, which may reduce an average diameter of the particles, the probability of hydrogen bonding of hyaluronic acid within the polymer chain increases, which allows for the manufacture of hyaluronic acid that is micronized to a smaller size. Accordingly, the composition according to an embodiment of the present disclosure facilitates the penetration of the high molecular weight hyaluronic acid particles into the skin, resulting in increased skin penetration rate and depth of penetration, and therefore enables delivery of the high molecular weight hyaluronic acid to the lower stratum corneum upon dermal administration. Additionally, according to an embodiment of the present disclosure, the micronized high molecular weight hyaluronic acid particles exhibit pH sensitivity. Therefore, after penetrating the skin, they may be neutralized in the skin's pH environment, for example, at a pH of 5 to 7, and more specifically, at approximately pH 5 to 6, without requiring a separate two-component formulation. As a result, the size of the high molecular weight hyaluronic acid particles may expand by 200% or more, and more specifically, by 200% or more, 210% or more, 250% or more, 300% or more, 350% or more, 400% or more, or 450% or more. The micronized high molecular weight hyaluronic acid particles may provide beneficial effects to the skin, such as moisturizing effects within the skin, prevention or improvement of skin wrinkles, prevention of skin elasticity reduction, or improvement of skin elasticity due to their self-swelling properties. Further, the composition comprising the micronized high molecular weight hyaluronic acid particles according to an embodiment of the present disclosure maintains a stable formulation even at a high temperature, thereby solving the problem in that the conventional composition comprising high molecular weight hyaluronic acid has broken formulation and become less viscous due to high temperature treatment during an aging process for productization. In an embodiment, the composition may provide excellent penetration into the skin and formulation stability of high molecular weight hyaluronic acid through micronization, without the need for polyhydric alcohols or solubilizing agents.

In an embodiment, the micronized high molecular weight hyaluronic acid particles may have an average particle diameter of 100 nm or less. As used in the present specification, the particle diameter of the particle means the largest diameter in the particle, and an average of the particle diameters means an average of the particle diameters of at least 90% or more of the particles distributed in the composition. Specifically, the average particle diameter of the particles, that is, an average of particle diameters, may mean an average value of the largest diameter in a particle of at least 90% or more, 91% or more, 92% or more, 93% or more, 94% or more, 95% or more, 96% or more, 97% or more, 98% or more, or 99% or more of the particles distributed in the composition. More specifically, the average particle diameter may be greater than 0 nm but 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, 25 nm or less, 20 nm or less, 15 nm or less, or 10 nm or less.

In view of the micronization of high molecular weight hyaluronic acid particles as described above, the pH of the composition according to an embodiment may be 3.0 or more but less than 5.0. Specifically, the pH of the composition may be 3 or more, 3.1 or more, 3.2 or more, 3.3 or more, 3.4 or more, 3.5 or more, 3.6 or more, 3.7 or more, 3.8 or more, 3.9 or more, 4.0 or more, 4.1 or more, 4.2 or more, 4.3 or more, 4.4 or more, 4.5 or more, 4.6 or more, 4.7 or more, 4.8 or more, or 4.9 or more, and may be less than 5.0, 4.9 or less, 4.8 or less, 4.7 or less, 4.6 or less, 4.5 or less, 4.4 or less, 4.3 or less, 4.2 or less, 4.1 or less, 4.0 or less, 3.9 or less, 3.8 or less, 3.7 or less, 3.6 or less, 3.5 or less, 3.4 or less, 3.3 or less, 3.2 or less, or 3.1 or less. When the pH of the composition according to an embodiment is less than 3.0, the composition may be irritating to the skin and is therefore not suitable for formulation into a cosmetic composition according to distribution cosmetic safety control standards. When the pH is 5.0 or more, the high molecular weight hyaluronic acid particles may not be able to be micronized to an average particle diameter of 100 nm or less.

In an embodiment, the acidic pH adjuster may be organic or inorganic acid, and the type thereof is not limited, as long as the acidic pH adjuster is capable of adjusting the pH of the composition to 3.0 or more but less than 5.0. For example, the organic acid may comprise one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, gluconolactone, and the like. For example, the inorganic acid may comprise one or more species selected from the group consisting of carbonic acid, hydrochloric acid, nitric acid, sulfuric acid, and the like.

In an embodiment, the acidic pH adjuster may comprise a fruit extract comprising the organic acid or inorganic acid described above, and may be used without limitation for the purpose of lowering pH in skincare products, whether or not the acidic pH adjuster is within the scope thereof. For example, the fruit extract may comprise one or more species selected the group consisting of Vaccinium myrtillus fruit extract, Saccharum officinarum (sugar cane) extract, citrus aurantium dulcis (orange) fruit extract, and Citrus limon (lemon) fruit extract, and the like.

In an embodiment of the present disclosure, the organic or inorganic acid is not comprised in the form of a salt, but is comprised as an acid itself. Unlike, for example, when a metal salt of an organic acid is comprised, where the metal salt acts as a metal cation, a hydrogen cation of the acid may interact with a negative charge of the high molecular weight hyaluronic acid to adjust the pH of the composition to less than 5.0. Accordingly, the high molecular weight hyaluronic acid may be micronized such that the average diameter of the particle is 100 nm or less.

In an embodiment, a weight ratio of the high molecular weight hyaluronic acid or salt thereof to the acidic pH adjuster may be 1:0.01 to 10. Specifically, the acidic pH adjuster may comprise 0.01 parts by weight or more, 0.1 parts by weight or more, 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, 1 part by weight or more, 1.1 parts by weight or more, 1.2 parts by weight or more, 1.3 parts by weight or more, 1.4 parts by weight or more, 1.5 parts by weight or more, 1.6 parts by weight or more, 1.7 parts by weight or more, 1.8 parts by weight or more, 1.9 parts by weight or more, 2 parts by weight or more, or 5 parts by weight or more based on 1 part by weight of the high molecular weight hyaluronic acid or salt thereof, and may comprise 10 parts by weight or less, 9 parts by weight or less, 8 parts by weight or less, 7 parts by weight or less, 6 parts by weight or less, 5 parts by weight or less, 4 parts by weight or less, 3 parts by weight or less, 2 parts by weight or less, 1.9 parts by weight or less, 1.8 parts by weight or less, 1.7 parts by weight or less, 1.6 parts by weight or less, 1.5 parts by weight or less, 1.4 parts by weight or less, 1.3 parts by weight or less, 1.2 parts by weight or less, 1.1 parts by weight or less, 1 parts by weight or less, 0.9 parts by weight or less, 0.8 parts by weight or less, 0.7 parts by weight or less, 0.6 parts by weight or less, 0.5 parts by weight or less, or 0.1 parts by weight or less based on 1 part by weight of the high molecular weight hyaluronic acid or salt thereof. When the weight ratio of the acidic pH adjuster to the high molecular weight hyaluronic acid or salt thereof is out of the range above, the particles of the high molecular weight hyaluronic acid or salt thereof may not be micronized or the pH may be too low below 3.0, resulting in skin irritation.

In an embodiment, a content of the high molecular weight hyaluronic acid or salt thereof may be 0.1 to 10 wt % based on a total weight of the composition. For example, the content of the high molecular weight hyaluronic acid or salt thereof may be 0.1 wt % or more, 0.3 wt % or more, 0.5 wt % or more, 0.6 wt % or more, 0.7 wt % or more, 0.8 wt % or more, 0.9 wt % or more, 1.0 wt % or more, 1.1 wt % or more, 1.2 wt % or more, 1.3 wt % or more, 1.5 wt % or more, 1.7 wt % or more, 2 wt % or more, 3 wt % or more, 4 wt % or more, 5 wt % or more, 6 wt % or more, 7 wt % or more, 8 wt % or more, or 9 wt % or more, and may be 10 wt % or less, 9 wt % or less, 8 wt % or less, 7 wt % or less, 6 wt % or less, 5 wt % or less, 4 wt % or less, 3 wt % or less, 2.5 wt % or less, 2 wt % or less, 1.5 wt % or less, 1.3 wt % or less, 1.2 wt % or less, 1.1 wt % or less, 1 wt % or less, 0.9 wt % or less, or 0.8 wt % or less, based on a total weight of the composition. When the content is less than 0.1 wt %, the effect on the skin may be negligible, and when the content is greater than 10 wt %, the content may be excessive relative to the acidic pH adjuster, which may prevent the particles of the high molecular weight hyaluronic acid or salt thereof from being micronized, and cause the viscosity to be too high to be manufactured into a liquid formulation.

In an embodiment, a content of the acidic pH adjuster may be 0.01 to 50 wt % based on the total weight of the composition. For example, the content of the acidic pH adjuster may be 0.01 wt % or more, 0.05 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1.0 wt % or more, 1.1 wt % or more, 1.2 wt % or more, 1.3 wt % or more, 1.4 wt % or more, 1.5 wt % or more, 2 wt % or more, 3 wt % or more, 4 wt % or more, 5 wt % or more, 10 wt % or more, 15 wt % or more, 20 wt % or more, 25 wt % or more, 30 wt % or more, 35 wt % or more, 40 wt % or more, or 45 wt % or more, and may be 50 wt % or less, 45 wt % or less, 35 wt % or less, 30 wt % or less, 25 wt % or less, 20 wt % or less, 15 wt % or less, 10 wt % or less, 9 wt % or less, 8 wt % or less, 7 wt % or less, 6 wt % or less, 5 wt % or less, 4 wt % or less, 3 wt % or less, 2 wt % or less, 1.5 wt % or less, 1.4 wt % or less, 1.3 wt % or less, 1.2 wt % or less, 1.1 wt % or less, 1 wt % or less, 0.9 wt % or less, 0.8 wt % or less, 0.7 wt % or less, 0.6 wt % or less, 0.5 wt % or less, 0.4 wt % or less, or 0.1 wt % or less, based on a total weight of the composition. When the content is less than 0.01 wt %, the particles of the high molecular weight hyaluronic acid or salt thereof may not be micronized, and when the content is more than 50 wt %, skin irritation may occur.

In an embodiment, the composition of the present disclosure may further comprise a metal ion sequestering agent. The metal ion sequestering agent, also referred to as a chelating agent, is generally used to bind to and inactivate metallic ions such as calcium, magnesium, or copper that adversely affect the stability or properties of products such as cosmetics. When the organic acid or inorganic acid is used in the form of a metal salt or the like as the acidic pH adjuster for the micronization of the high molecular weight hyaluronic acid particles, it is difficult to penetrate the skin because the high molecular weight hyaluronic acid particles swell in the composition and are not micronized when the composition comprises the metal ion sequestering agent as described above. In contrast, in an embodiment of the present disclosure, since the organic acid or inorganic acid is comprised as the acid itself rather than in the form of a metal salt or the like, it is possible to micronize the high molecular weight hyaluronic acid particles to an average diameter of 100 nm or less because the micronization is not impeded even if the metal ion sequestering agent is further comprised in order to improve the stability and usability of the products. In addition, since the composition according to the present disclosure reduces the pH to less than 5.0 to micronize the high molecular weight hyaluronic acid particles to penetrate the skin, the pH of the high molecular weight hyaluronic acid particles naturally increases to 5.5 to 6.0 and swells due to a difference in acidity from the natural pH of the skin, thereby increasing in particle size without the administration of additional ingredients.

In an embodiment, the metal ion sequestering agent is not limited in type, but may comprise, for example, one or more species of an alkaline salt of ethylene-diamine-tetraacetic acid (EDTA) and an alkaline salt of a phosphate-based compound. More specifically, the metal ion sequestering agent may comprise one or more species selected from the group consisting of disodium EDTA (ethylene-diamine-tetraacetic acid), tetrasodium EDTA, trisodium ethylenediamine disuccinate, sodium phosphate, monosodium phosphate, disodium phosphate, trisodium phosphate, sodium tripolyphosphate, sodium hexametaphosphate, sodium metaphosphate, sodium phytate, tetrasodium pyrophosphate, and sodium acid pyrophosphate. In addition, a content of the metal ion sequestering agent according to an embodiment may be 0.01 to 1 wt % based on the total weight of the composition. Specifically, the content of the metal ion sequestering agent may be 0.001 wt % or more, 0.005 wt % or more, 0.01 wt % or more, 0.02 wt % or more, 0.03 wt % or more, 0.04 wt % or more, 0.05 wt % or more, 0.1 wt % or more, 0.5 wt % or more, or 0.9 wt % or more, and may be 1 wt % or less, 0.9 wt % or less, 0.8 wt % or less, 0.7 wt % or less, 0.6 wt % or less, 0.5 wt % or less, 0.4 wt % or less, 0.3 wt % or less, 0.2 wt % or less, 0.1 wt % or less, 0.05 wt % or less, 0.04 wt % or less, 0.03 wt % or less, or 0.01 wt % or less, based on a total weight of the composition. When the content of the metal ion sequestering agent is less than 0.01 wt %, the sequestering effect may be less, and when the content is more than 1 wt %, the negative effect on the skin may occur by sequestering even the beneficial metal ions in the skin cells.

In an embodiment, the composition may provide excellent penetration into the human body and formulation stability of high molecular weight hyaluronic acid through micronization, without the need for polyhydric alcohols or solubilizing agents.

In an embodiment, viscosity or hardness of the composition may vary depending on the formulation of the composition. For example, the viscosity may be 0.001 to 50 Pa·s, but is not limited thereto. For example, the hardness may be 10 to 200 N, but is not limited thereto.

The present disclosure may provide a method of manufacturing the composition according to the embodiments described above, the method comprises: micronizing a high molecular weight hyaluronic acid or salt thereof by adding an acidic pH adjuster to a solution that comprises the high molecular weight hyaluronic acid or salt thereof, in which the micronized high molecular weight hyaluronic acid or salt particle thereof has an average particle diameter of 100 nm or less, and a pH of the composition is 3.0 or more but less than 5.0.

As an embodiment, the method may further comprise adding a metal ion sequestering agent to the solution at the micronizing of the high molecular weight hyaluronic acid or salt thereof.

As an embodiment, the method may further comprise dissolving the high molecular weight hyaluronic acid or salt thereof in a solution that comprises one or more of water and an organic solvent prior to the micronizing of the high molecular weight hyaluronic acid or salt thereof.

As an embodiment, the micronizing of the high molecular weight hyaluronic acid or salt thereof may further comprise stirring and dissolving the solution using a magnetic stirrer.

As an embodiment, the method may further comprise aging the composition at a temperature of 30 to 60° C. after the micronizing of the high molecular weight hyaluronic acid or salt thereof. In this case, the aging time according to an embodiment is not limited, but may be, for example, 5 to 20 hours.

In an embodiment, the composition may be for moisturizing the skin. In an embodiment, the composition may be for preventing loss of skin elasticity or improving skin elasticity. In an embodiment, the composition may be for preventing the formation of skin wrinkles or for improving skin wrinkles. Another embodiment of the present disclosure may provide a method of moisturizing skin, preventing loss of skin elasticity or improving skin elasticity, or preventing the formation of skin wrinkles or improving skin wrinkles, the method comprises administering an effective amount of a composition to the skin of a subject in need thereof, in which the composition comprises: a high molecular weight hyaluronic acid or salt particles thereof; and an acidic pH adjuster, in which the acidic pH adjuster is an organic acid or inorganic acid, the high molecular weight hyaluronic acid or salt particles thereof have an average particle diameter of 100 nm or less, and a pH of the composition is 3.0 or more but less than 5.0. Still another embodiment may provide a use of a high molecular weight hyaluronic acid or salt particle thereof; and an acidic pH adjuster, for manufacturing a composition for moisturizing skin, preventing loss of skin elasticity, improving skin elasticity, preventing the formation of skin wrinkles, or improving skin wrinkles. Still another embodiment may provide a high molecular weight hyaluronic acid or salt particles thereof; and an acidic pH adjuster, for using in a composition for moisturizing skin, preventing loss of skin elasticity, improving skin elasticity, preventing the formation of skin wrinkles, or improving skin wrinkles. Yet another embodiment may provide a non-therapeutic cosmetic use of a high molecular weight hyaluronic acid or salt particle thereof; and an acidic pH adjuster as an effective ingredient for moisturizing skin, preventing loss of skin elasticity, improving skin elasticity, preventing the formation of skin wrinkles, or improving skin wrinkles. In this case, the acidic pH adjuster is an organic acid or inorganic acid, an average particle diameter of the high molecular weight hyaluronic acid or salt particles thereof is 100 nm or less, and a pH of the composition may be 3.0 or more but less than 5.0.

In an embodiment, the composition may be a skin external application composition. As used in the present disclosure, the term “skin external application composition” means a composition of a formulation that is applied externally to the skin. As an embodiment, the skin external application composition may be applied by hand or using various application means, such as a spatula, and the like, and may include various formulations of cosmetics, pharmaceuticals, non-pharmaceuticals, and the like.

Additionally, the composition according to embodiments of the present disclosure may be provided as a carrier composition for transdermal delivery of an active substance, having pH sensitivity that enables the release of the active substance at a pH of 5 to 7.

In the present disclosure, the term “pH sensitivity” refers to changes in the characteristics of a substance depending on pH. Specifically, in the present disclosure, it means that the size and shape of the high molecular weight hyaluronic acid particles, as well as their characteristics to capture or release active substances, change in response to pH variations.

In an embodiment, the carrier composition may penetrate the skin in a form where the active substance is physically captured through electrostatic interactions with micronized high molecular weight hyaluronic acid particles. Subsequently, responding to the skin's pH, the particles may naturally expand in size, effectively releasing and delivering the captured active substance into the skin. The carrier composition facilitates the penetration of high molecular weight hyaluronic acid particles into the skin, thereby improving skin permeability and penetration depth. As a result, when applied to the skin, it may enhance the skin absorption of the active substance into the lower stratum corneum.

From this perspective, another embodiment of the present disclosure may provide a skin external application composition comprising: an active substance; and a carrier for the transdermal delivery of the active substance, comprising the aforementioned composition.

Another embodiment of the present disclosure may provide a use of the micronized high molecular weight hyaluronic acid or salt particles thereof for use in the manufacture of a carrier composition for transdermal delivery of the active substance. Another embodiment may provide a method for transdermal delivery of an active substance, comprising: capturing an effective amount of the active substance in the micronized high molecular weight hyaluronic acid or salt particles thereof. Another embodiment may provide the micronized high molecular weight hyaluronic acid or salt thereof for use as a carrier for transdermal delivery of an active substance. Another embodiment may provide a use of the micronized high molecular weight hyaluronic acid or salt thereof as a carrier for transdermal delivery of an active substance. Another embodiment may provide a non-therapeutic use of the micronized high molecular weight hyaluronic acid or salt thereof as a carrier for transdermal delivery.

As an embodiment, the active substance may comprise any substance that has beneficial effects on the skin or human body, without being limited by its effectiveness or the type of substance. As an embodiment, the active substance may be a substance that is unstable and prone to degradation, or a substance that is poorly soluble or insoluble, making transdermal penetration difficult by itself. As an embodiment, the active substance may be active substances commonly used in the cosmetics or pharmaceutical fields. As an embodiment, the active substance may comprise one or more of organic acids or derivatives thereof. For example, the organic acid may comprise one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, gluconolactone, and the like. As an embodiment, the organic acid derivatives may comprise, for example, 3-O-ethyl ascorbic acid, ascorbyl glucoside, sodium ascorbyl phosphate, and the like.

As an embodiment, the active substance may be comprised in an amount of 0.01 to 40 wt % based on the total weight of the skin external application composition. Specifically, the active substance may be comprised in an amount of0.01 wt % or more, 0.02 wt % or more, 0.03 wt % or more, 0.04 wt % or more, 0.05 wt % or more, 0.06 wt % or more, 0.07 wt % or more, 0.08 wt % or more, 0.09 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1 wt % or more, 2 wt % or more, 3 wt % or more, 4 wt % or more, 5 wt % or more, 6 wt % or more, 7 wt % or more, 8 wt % or more, 9 wt % or more, 10 wt % or more, 15 wt % or more, 20 wt % or more, 25 wt % or more, or 29 wt % or more, based on the total weight of the skin external application composition. As an embodiment, the active substance may be comprised in an amount of 30 wt % or less, 25 wt % or less, 20 wt % or less, 15 wt % or less, 10 wt % or less, 9 wt % or less, 8 wt % or less, 7 wt % or less, 6 wt % or less, 5 wt % or less, 4 wt % or less, 3 wt % or less, 2 wt % or less, 1 wt % or less, 0.9 wt % or less, 0.8 wt % or less, 0.7 wt % or less, 0.6 wt % or less, 0.5 wt % or less, 0.4 wt % or less, 0.3 wt % or less, 0.2 wt % or less, 0.1 wt % or less, 0.09 wt % or less, 0.08 wt % or less, 0.07 wt % or less, 0.06 wt % or less, 0.05 wt % or less, 0.04 wt % or less, 0.03 wt % or less, 0.02 wt % or less, or 0.01 wt % or less, based on the total weight of the skin external application composition. Additionally, as an embodiment, the weight ratio of the carrier composition to the total weight of the active substance may vary depending on the type of the active substance. For example, the weight ratio may be 1:0.01 to 100. Specifically, the weight ratio of the carrier composition to the total weight of the active substance may be 1:0.01 or more, 0.1 or more, 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, or 90 or more, and may be 100 or less, 90 or less, 80 or less, 70 or less, 60 or less, 50 or less, 40 or less, 30 or less, 20 or less, 10 or less, 9 or less, 8 or less, 7 or less, 6 or less, 5 or less, 4 or less, 3 or less, or 2 or less. When the active substance is comprised in insufficient amounts, its intended effectiveness may not be fully realized. On the other hand, when the active substance is excessively comprised, the content of the carrier composition may decrease relatively, leading to a reduction in skin delivery efficiency.

As an embodiment, the composition may further comprise a basic pH adjuster, in addition to the acidic pH adjuster, to adjust the pH of the composition to be 3.0 or more but less than 5.0, depending on the type and content of the active substance.

Additionally, in an embodiment, the skin external application composition may be provided for various uses depending on the type of active substance. For example, the composition may be for moisturizing skin. In an embodiment, the composition may be for preventing loss of skin elasticity or improving skin elasticity. In an embodiment, the composition may be for preventing the formation of skin wrinkles or for improving skin wrinkles. In an embodiment, the composition may be for whitening skin. In an embodiment, the composition may be for anti-inflammatory purposes. In an embodiment, the composition may be for the prevention, improvement, or treatment of acne. Another embodiment of the present disclosure may provide a method for skin moisturizing, skin whitening, prevention of skin elasticity reduction or improvement of skin elasticity, prevention of skin wrinkle formation or skin wrinkle improvement, anti-inflammatory, or anti-acne treatment, comprising: administering an effective amount of the carrier composition that captures the aforementioned active substance to the skin of a subject in need thereof. Still another embodiment may provide a use of the carrier composition that captures the aforementioned active substance for manufacturing a skin external application composition for skin moisturizing, skin whitening, prevention of skin elasticity reduction or improvement of skin elasticity, prevention of skin wrinkle formation or skin wrinkle improvement, anti-inflammatory, or anti-acne treatment. Still another embodiment may provide a carrier composition that captures the aforementioned active substance for use in compositions for skin moisturizing, skin whitening, prevention of skin elasticity reduction or improvement of skin elasticity, prevention of skin wrinkle formation or skin wrinkle improvement, anti-inflammatory, or anti-acne treatment. Yet another embodiment may provide the non-therapeutic cosmetic use of a carrier composition that captures the aforementioned active substance as an effective ingredient for skin moisturizing, skin whitening, prevention of skin elasticity reduction or improvement of skin elasticity, prevention of skin wrinkle formation or skin wrinkle improvement, anti-inflammatory, or anti-acne treatment.

In an embodiment, the composition may be a cosmetic composition. In an embodiment, the appearance of the cosmetic composition may comprise a medium or a base that is cosmetologically or dermatologically acceptable. For example, the appearance of the cosmetic composition may be provided in the form of a solution, gel, solid, pasty anhydrous product, emulsion obtained by dispersing an oil phase in an aqueous phase, suspension, microemulsion, microcapsule, microgranule, or ionic (liposome) and non-ionic vesicular dispersions, or in the form of a cream, skin, lotion, powder, ointment, spray, or conceal stick. These compositions may be manufactured according to conventional methods in the art. The composition according to the present disclosure may also be used in the form of a foam or in the form of an aerosol composition further comprising a compressed propellant.

The cosmetic composition according to an embodiment of the present disclosure is not particularly limited in the formulation and may be formulated into cosmetic products such as, for example, an ampoule, cream, softening cosmetic water, astringent cosmetic water, nutritional cosmetic water, nutritional cream, gel cream, massage cream, essence, eye cream, eye essence, cleansing cream, cleansing foam, cleansing water, and a cleansing tissue, pack, body lotion, body cream, body oil, and body essence that comprise the cosmetic compositions. When the formulation according to an embodiment of the present disclosure is a paste, cream or gel, an animal fiber, plant fiber, wax, paraffin, starch, tragacanth, cellulose derivative, polyethylene glycol, silicone, bentonite, silica, talc or zinc oxide may be used as a carrier component. When the formulation according to an embodiment of the present disclosure is a spray, a lactose, talc, silica, aluminum hydroxide, calcium silicate, or polyamide powder may be used as a carrier component, and a propellant such as chlorofluorohydrocarbon, propane/butane, or dimethyl ether may be further comprised. When the formulation according to an embodiment of the present disclosure is a solution or emulsion, a solvent, solubilizer or emulsifier is used as a carrier component, for example, there are water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan. When the formulation according to an embodiment of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum methahydroxide, bentonite, agar or tragacanth may be used as a carrier component. When the formulation according to an embodiment of the present disclosure is a surfactant-containing cleansing, aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkylamidobetaine, aliphatic alcohol, fatty acid glyceride, fatty acid diethanolamide, vegetable oil, linolein derivatives or ethoxylated glycerol fatty acid ester may be used as a carrier component.

The cosmetic composition according to an embodiment of the present disclosure may further comprise functional additives and ingredients comprised in a general cosmetic composition. The functional additives may comprise ingredients selected from the group consisting of a water-soluble vitamin, oil-soluble vitamin, polymer peptide, polymer polysaccharide, sphingolipid, and seaweed extract. In addition, blending ingredients that may be comprised in the cosmetic composition comprise a retaining moisture ingredient, moisturizer, emollient, neutralizer, surfactant, organic and inorganic pigment, organic powder, UV absorber, preservative, bactericide, antioxidant, plant extract, pH adjuster, alcohol, pigment, fragrance, blood flow stimulant, cooling agent, antiperspirant, purified water, and the like.

In an embodiment, the composition may be a pharmaceutical composition. When the composition according to an embodiment of the present disclosure is applied to a pharmaceutical product, the effective ingredient used in an embodiment of the present disclosure may be added to an inorganic or organic carrier that is commonly available and formulated into a semi-solid or liquid form of non-oral administration, and the pharmaceutical composition according to the present disclosure may be administered non-orally, rectally, topically, transdermally, intravenously, intramuscularly, intraperitoneally, subcutaneously, and the like. In an embodiment, the pharmaceutical composition may be a non-oral agent, more specifically a skin external application agent. For example, the skin external application agent may be a formulation such as an ointment, skin, lotion, gel, cream, spray, suspension, emulsion, patch, or the like, but is not limited thereto. The composition according to an embodiment of the present disclosure may be manufactured according to commonly known methods in the art to facilitate the formulation of the effective ingredient, in which case surfactants, excipients, colorants, fragrances, preservatives, stabilizers, buffers, suspending agents, and other commonly available supplements may be appropriately used. A dosage amount of the effective ingredient of the pharmaceutical composition according to an embodiment of the present disclosure will depend on an age, sex, weight, pathologic state and severity of a subject to be administered, a route of administration, or judgment of a prescriber. A determination of the appropriate dosage amount based on these factors is within the skill of those skilled in the art and a daily dosage amount may be, for example, 0.1 to 100 mg/kg/day, and more specifically, 5 to 50 mg/kg/day, but is not limited thereto.

In an embodiment, the composition may be a food composition. The food composition according to an embodiment of the present disclosure may be a formulation in a liquid or solid state, and may be a formulation of a tablet, capsule, soft capsule, iozenge, granule, beverage (drink), diet bar, chocolate, caramel formulation, or snack, but is not particularly limited thereto. In addition to the effective ingredients above, the food composition according to an embodiment of the present disclosure may comprise excipients, sugars, flavors, colors, oils, proteins, and the like as needed.

### MODE FOR CARRYING OUT INVENTION

Hereinafter, the present disclosure will be described in more detail with reference to the following embodiments. However, the following embodiments are provided for illustrative purposes only to assist in understanding the present disclosure and are not intended to limit the scope and range of the present disclosure.

### Test Example 1

To confirm the micronization of the high molecular weight hyaluronic acid or salt thereof according to the present disclosure, as an embodiment, Examples 1 to 4 having the composition of Table 1 below were manufactured by the following method.

First, sodium hyaluronate (manufacturer: Hyundai Bioland, product name: Bio sodium hyaluronate, weight average molecular weight: 1,500 KDa) was prepared as a high molecular weight hyaluronic acid salt. In Examples 1 to 4, 1,2-hexanediol and sodium hyaluronate were dissolved in deionized water using a magnetic stirrer, and then the remaining ingredients were added and mixed with a homogenizer at a high speed of 12000 rpm to induce particle micronization. In this case, the pH of the micronized solution was in the range of 3 to 4. After the hyaluronic acid solution was input into the 45° C. chamber, a final composition was manufactured by stabilizing the particles and properties through an aging process for 12 hours or more.

As a comparative example to compare the effects of the examples above, Comparative Example 1, which comprises only a solvent and sodium hyaluronate, and Comparative Example 2, which comprises trisodium citrate (pH 6.5) that is a type of conjugate base, that is, a metal salt of an organic acid, as a pH adjuster, were manufactured in the same manner as Examples 1 to 4 above.

TABLE 1

| | | | | | Comparative | Comparative |
|---|---|---|---|---|---|---|
| Components | Example 1 | Example 2 | Example 3 | Example 4 | Example 1 | Example 2 |
| Deionized water | To 100 | To 100 | To 100 | To 100 | To 100 | To 100 |
| 1,2-Hexanediol | 1.2 | 1.2 | 1.2 | 1.2 | 1.2 | 1.2 |
| Sodium | 0.8 | 0.8 | 0.8 | 0.8 | 0.8 | 0.8 |
| hyaluronate | | | | | | |
| Ascorbic acid | 0.9 | — | — | 0.9 | — | — |
| Succinic acid | — | 1.5 | — | — | — | — |
| Citric Acid | — | — | 0.4 | — | — | — |
| Trisodium citrate | — | — | — | — | — | 0.5 |
| Disodium EDTA | — | — | — | 0.03 | — | — |

(wt %)

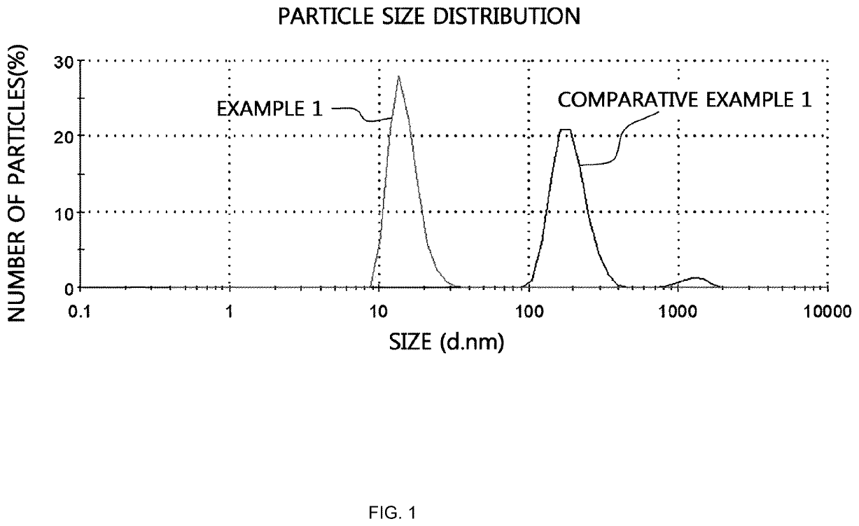

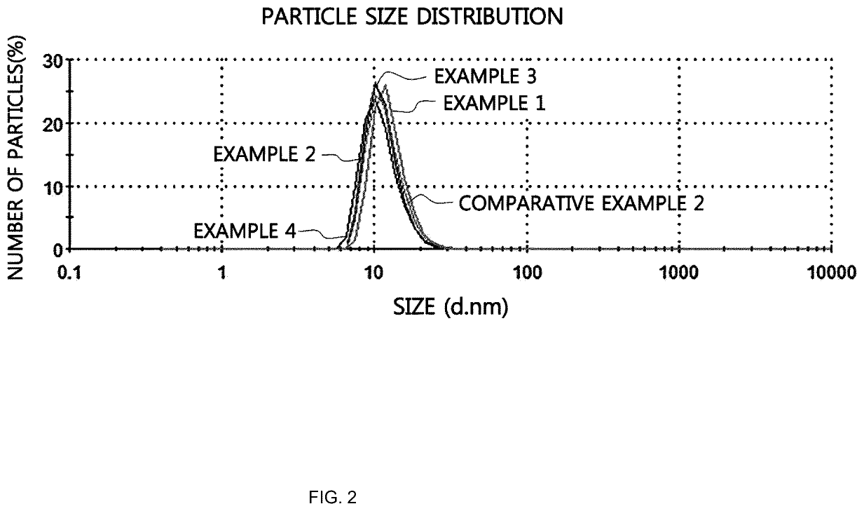

To compare the micronization of the high molecular weight hyaluronic acids or salts thereof of Examples 1 to 4 with that of Comparative Examples 1 and 2, the average particle diameter of each composition was measured using a Zetasizer instrument from Malvern Panalytical company, and is illustrated in FIGS. 1 and 2.

As a result, as illustrated in FIGS. 1 and 2, it can be seen that the particles of the high molecular weight hyaluronic acid salt of Comparative Example 1, which is an aqueous solution of high molecular weight hyaluronic acid without the addition of the pH adjuster, were distributed in the range of 100 nm to 1000 nm and did not decrease in particle size, but the particles of the high molecular weight hyaluronic acid salt of Examples 1 to 4 were all distributed in the range of 10 nm to 100 nm in size even with the change of acid type and the addition of the metal ion sequestering agent, and were micronized to about 10% or less of that of Comparative Example 1. In Comparative Example 2, the average particle size of the high molecular weight hyaluronic acid salt was found to be 100 nm or less, but there is a limitation that the particle size does not expand again after being administered to the skin by the metal salt of the organic acid being used as the pH adjuster, as confirmed in Test Example 2 described below.

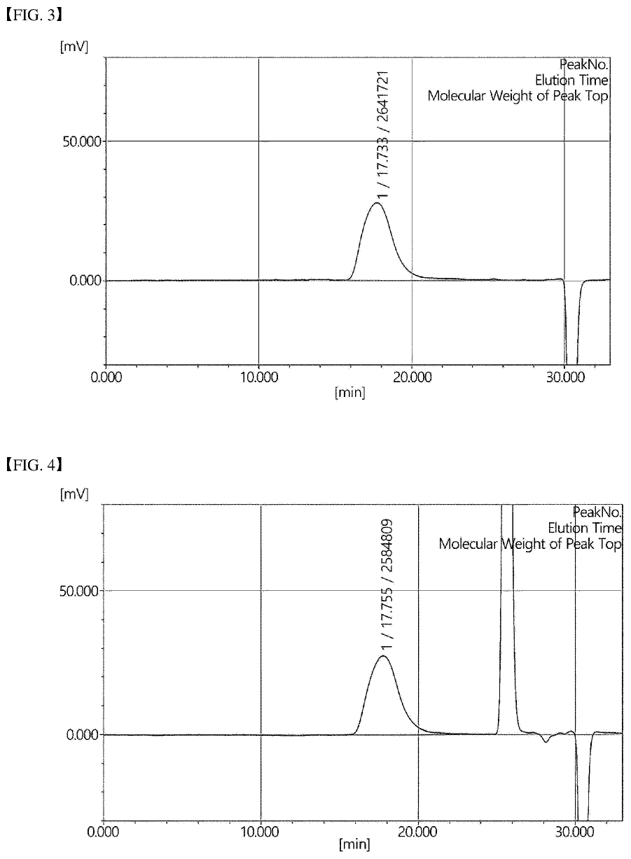

In addition, as a result of measuring relative molecular weights of Example 1 and Comparative Example 1 by gel chromatography (GPC) requested to Korea Polymer Testing Institute (Koptri), an accredited testing agency, it was found that both high molecular weight hyaluronic acid salts of Comparative Example 1 (FIG. 3) and Example 1 (FIG. 4) showed the same peak at 17 minutes, indicating that the maximum molecular weights were the same. The results above mean that in Example 1, the high molecular weight hyaluronic acid salt was micronized to a particle size of 100 nm or less without changing the molecular weight of the high molecular weight hyaluronic acid salt.

### Test Example 21

It was confirmed whether the high molecular weight hyaluronic acid or salt particle thereof that are micronized according to the present disclosure are capable of providing the effect of the high molecular weight hyaluronic acid salt on the skin, such as moisturizing the skin or improving skin elasticity, by the high molecular weight hyaluronic acid or salt particle thereof being administered to the skin, neutralized by the pH of the skin, and then expanded in size again, through a change in the particle size of the micronized high molecular weight hyaluronic acid or salt particle thereof in response to a change in pH, with respect to Examples 1 to 4 manufactured in Test Example 1 and Comparative Example 2.

Specifically, a base neutralizer (manufacturer: ANGUS, product name: Tromethamine) was added to each of Examples 1 to 4 and Comparative Example 2 manufactured in Test Example 1 above to increase the pH of each composition to pH 6, which is the same as the pH of the skin, and then a change in particle behavior was confirmed using Zetasizer (Malvern, UK) instrument.

As a result, as shown in Table 2 below, it was confirmed that for Examples 1 to 4, the particle size increased again as the pH of the composition increased compared to before the pH increased, but for Comparative Example 2, the change in particle size was insignificant and rather the particle size slightly decreased.

TABLE 2

| | | | | | Comparative |
|---|---|---|---|---|---|
| | Example 1 | Example 2 | Example 3 | Example 4 | Example 2 |
| Average | 460% | 390% | 210% | 250% | −10% |
| particle | | | | | |
| diameter | | | | | |
| increase | | | | | |
| rate (%) | | | | | |

### Test Example 31

A change in physical properties of the composition according to the present disclosure was confirmed by viscosity measurements with respect to Examples 1 to 4 and Comparative Example 1, manufactured in Test Example 1.

In this case, viscosity (Pa·s) was measured using a rheometer (Malvern, UK), oscillation stress adjusted to 1 Pa using plate type at 25° C.: CP 4/40, frequency range of 0.1-10 Hz, 1 Pa shear stress, and 1 mm gap are set conditions. Among those, G′ and G″ values at 1 Hz were measured, and the G′ and G″ measurement samples were standardized in the form of a disk with a diameter of 20 mm and a thickness of 1 mm.

As a result, as shown in Table 3 below, it can be seen that for Examples 1 to 4, even though the hyaluronic acid solution was aged at 45° C. for 12 hours or more during the manufacture, the physical properties of the composition were stabilized at a low viscosity.

TABLE 3

| | | | | | Comparative |
|---|---|---|---|---|---|
| | Example 1 | Example 2 | Example 3 | Example 4 | Example 1 |
| Viscosity | 0.023 | 0.025 | 0.018 | 0.022 | 0.306 |
| (Pa · s) | | | | | |

### Test Example 41

To confirm the increase in skin penetration by the micronization of the high molecular weight hyaluronic acid or salt particle thereof according to the present disclosure, the following experiment was conducted.

First, skin tissue of Fitzpatric type II (manufacturer: Genoskin, product name: NativeSkin) biopsied from the abdomen of a 33-year-old female was supplied by the manufacturer, and Example 1 and Comparative Example 1 manufactured in Test Example 1 were applied to the skin tissue, respectively. A tape disk (manufacturer: Cuderm, product name: D100, diameter: 22 mm) was attached to the skin tissue, pressed with a D-squame pressure device with a force of 225 gr·/sq·cm, and then the tape disk was removed. A total of 10 tape disks were sequentially removed at approximately 3-minute intervals, and the tape disk samples were stored in a freezer.

A desorption electrospray ionization (DESI) mass spectrometer (manufacturer: Waters, product name: 2D DESI stage & XEVO G2-XS QTof) was used to confirm whether each composition was permeable at each stage from tape disk samples stored in the freezer. In this case, DESI-MS spectra were acquired using the 2D DESI stage and XEVO G2-XS QTof (Waters, Milford, USA). A spray solvent was 98% methanol+0.1% formic acid, a flow rate was 2 μL/min, and a spray gas inlet pressure was set to 0.5 MPa. An area of 100×100 μm² was measured on the tape-stripped tape disk sample above. A mass range of 50 to 1,000 m/z was measured in a positive ion mode. MS imaging data were acquired using MassLynx 4.2 (Waters, USA) and visualized using High Definition Imaging (HDI) 1.5 software (Waters).

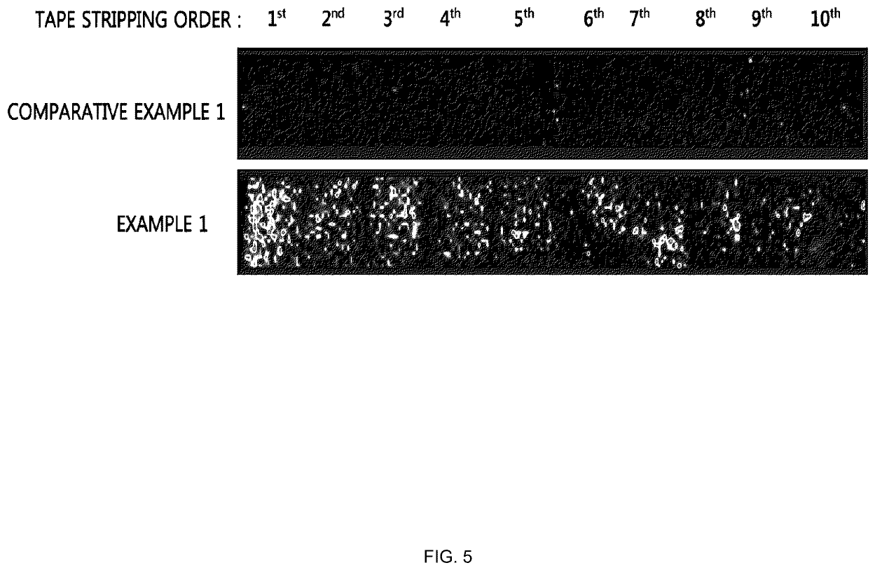

As a result, as illustrated in FIG. 5, for Comparative Example 1, it was not confirmed that any particles penetrated into the stratum corneum due to the large particle size of the high molecular weight hyaluronic acid salt, whereas for Example 1 according to an embodiment of the present disclosure, it was confirmed that there was deep penetration up to the 10^{th} order due to the micronization of the high molecular weight hyaluronic acid salt.

### Example 5

The carrier composition for transdermal delivery of the active substance and the composition comprising the active substance according to an embodiment of the present disclosure were manufactured using the following method.

First, sodium hyaluronate (manufacturer: Hyundai Bioland, product name: Bio sodium hyaluronate, weight average molecular weight: 1,500 KDa) was prepared as a high molecular weight hyaluronic acid salt. 1,2-hexanediol and the sodium hyaluronate were dissolved in deionized water using a magnetic stirrer. The remaining ingredients from Table 4 below were added, and while mixing with a homogenizer at 12,000 rpm, the particles were micronized to an average particle diameter of 100 nm or less. In this case, the pH of the micronized solution was in the range of 3 to 4. After the hyaluronic acid solution was input into the 45° C. chamber, a final composition was manufactured by stabilizing the particles and properties through an aging process for 12 hours or more.

### Comparative Example 3

As a comparative example for comparing the effects of the above example, Comparative Example 3 was manufactured using the same composition and method as in Example 5, except that it does not comprise sodium hyaluronate.

TABLE 4

| | | | Comparative |
|---|---|---|---|
| | Components | Example 5 | Example 3 |
| Solvent | Deionized water | To 100 | To 100 |
| Hyaluronic acid salt | Sodium hyaluronate | 0.6 | — |
| Metal ion sequestrant | Sodium metaphosphate | 0.08 | 0.08 |
| Active substance | Ascorbic acid | 0.5 | 0.5 |
| Stabilizer | Caffeine | 0.17 | 0.17 |
| Stabilizer | Mannitol | 0.1 | 0.1 |
| Preservative | 1,2-Hexanediol | 1.2 | 1.2 |
| Basic pH adjuster | Tromethamine | 0.3 | 0.3 |
| Acidic pH adjuster | Succinic acid | 0.5 | 0.5 |
| (wt %) | | | |

### Test Example 5

To confirm the transdermal delivery effect of the active substance by the carrier composition according to the present disclosure, the skin absorption amount of ascorbic acid, the active substance each in Example 5 and Comparative Example 3, was measured and compared using the following method.

The absorption amount of ascorbic acid was measured using a Franz diffusion cell. A membrane (Isopore, Merck Milipore, USA) was fixed between the donor and receptor phases, in which case the area in contact with the receptor phase was approximately 1.767 cm². Phosphate buffer saline (PBS, 7 mL) was used as the receptor phase, with constant stirring at 600 rpm while maintaining at 32° C. After 1 hour and 3 hours, the receptor phase was taken and the amount of ascorbic acid was quantified using high-performance liquid chromatography (HPLC). During the HPLC analysis, Mightysil RP-18 GP 250-4.6 (5 μm) was used as the stationary phase, and a mixed solution of 1% CH₃COOH:methanol (90:10, v/v) was used as the mobile phase. The detection was performed at a wavelength of 254 nm with a flow rate of 1 mL/min. The concentration was calculated from the area of the obtained peaks, according to the reference, “Snezana S Mitic, Danijela A Kostic, Danijela C Naskovic-Dokic, Milan N Mitic, Rapid and Reliable HPLC Method for the Determination of Vitamin C in Pharmaceutical Samples, Tropical Journal of Pharmaceutical Research February 2011; 10 (1): 105-111.” The above reference is hereby incorporated by reference in its entirety into this specification.

TABLE 5

| | After 1 hour | | After 3 hours | |
|---|---|---|---|---|
| Example 5 | 59.64 | μg/mL | 87.87 | μg/mL |
| Comparative Example 3 | 0 | μg/mL | 0 | μg/mL |

As a result, in Comparative Example 3, which is a composition comprising the active substance without the carrier composition of the present disclosure, the amount of ascorbic acid absorbed into the skin tissue was extremely low and not detectable. In contrast, it was confirmed that Example 5 demonstrated that ascorbic acid was effectively delivered into the skin tissue by the carrier composition comprising micronized high molecular weight hyaluronic acid salt, resulting in a high absorption amount.

### Test Example 6

To confirm the transdermal delivery effect of the active substance by the carrier composition according to the present disclosure, the penetration depth of the active substance into the skin was examined using the following method.

First, skin tissue of Fitzpatric type II (manufacturer: Genoskin, product name: NativeSkin) biopsied from the abdomen of a 33-year-old female was supplied by the manufacturer, and Example 5 and Comparative Example 3 were applied to the skin tissue, respectively. A tape disk (manufacturer: Cuderm, product name: D100, diameter: 22 mm) was attached to the skin tissue, pressed with a D-squame pressure device with a force of 225 gr·/sq·cm, and then the tape disk was removed. A total of 10 tape disks were sequentially removed at approximately 3-minute intervals, and the tape disk samples were stored in a freezer.

A desorption electrospray ionization (DESI) mass spectrometer (manufacturer: Waters, product name: 2D DESI stage & XEVO G2-XS QTof) was used to confirm whether each composition was permeable at each stage from tape disk samples stored in the freezer. In this case, DESI-MS spectra were acquired using the 2D DESI stage and XEVO G2-XS QTof (Waters, Milford, USA). A spray solvent was 98% methanol+0.1% formic acid, a flow rate was 2 μL/min, and a spray gas inlet pressure was set to 0.5 MPa. An area of 100×100 μm² was measured on the tape-stripped tape disk sample above. A mass range of 50 to 1,000 m/z was measured in a positive ion mode. MS imaging data were acquired using MassLynx 4.2 (Waters, USA) and visualized using High Definition Imaging (HDI) 1.5 software (Waters).

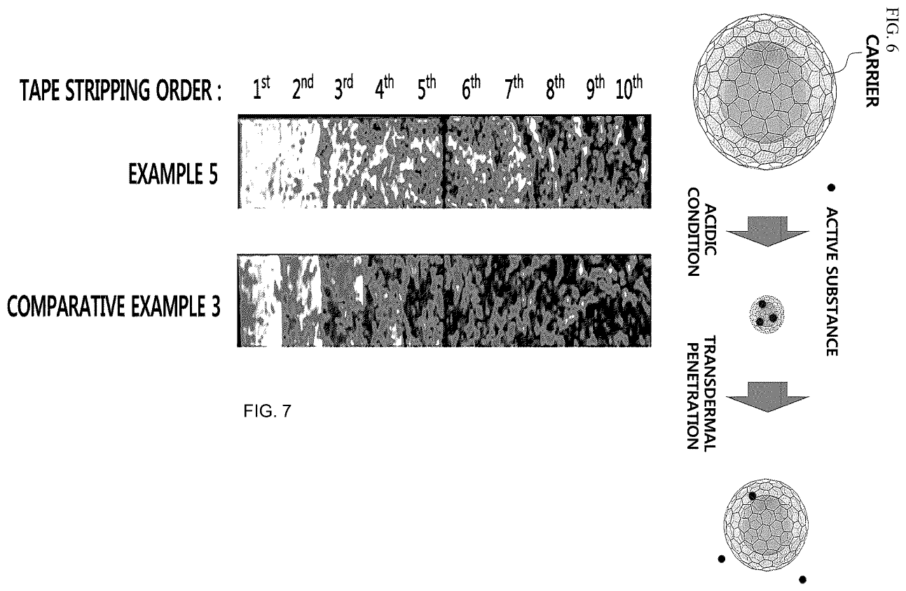

As a result, as illustrated in FIG. 7, in Comparative Example 3, which is a composition comprising the active substance without the carrier composition of the present disclosure, no active substance was detected as having penetrated the stratum corneum. In contrast, it was confirmed that Example 5 demonstrated that the active substance penetrated deeply up to the 10th tape stripping, by the carrier composition comprising micronized high molecular weight hyaluronic acid salt.

The present disclosure may provide the following embodiments as an example.

A first embodiment may provide a composition comprising: a high molecular weight hyaluronic acid or salt particles thereof; and an acidic pH adjuster, in which the acidic pH adjuster is an organic acid or inorganic acid, and in which the high molecular weight hyaluronic acid or salt particle thereof have an average particle diameter of 100 nm or less, and wherein a pH of the composition is 3.0 or more but less than 5.0.

A second embodiment may provide the composition of the first embodiment, which further comprises a metal ion sequestering agent.

A third embodiment may provide the composition of the first or second embodiment, in which the metal ion sequestering agent is one or more species of an alkaline salt of ethylene-diamine-tetraacetic acid (EDTA) and an alkaline salt of a phosphate-based compound.

A fourth embodiment may provide the composition of any one of the first to third embodiments, in which the high molecular weight hyaluronic acid or salt thereof has a weight average molecular weight of 5 to 10,000 kDa.

A fifth embodiment may provide the composition of any one of the first to fourth embodiments, in which a weight ratio of the high molecular weight hyaluronic acid or salt thereof to an acidic pH adjuster is 1:0.01 to 10.

A sixth embodiment may provide the composition of any one of the first to fifth embodiments, in which the high molecular weight hyaluronic acid or a salt thereof is comprised at 0.1 to 10 wt %, based on a total weight of the composition.

A seventh embodiment may provide the composition of any one of the first to sixth embodiments, in which the acidic pH adjuster is comprised at 0.01 to 50 wt %, based on the total weight of the composition.

An eighth-first embodiment may provide the composition of any one of the first to seventh embodiments, in which the organic acid is one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, and gluconolactone.

An eighth-second embodiment may provide the composition of any one of the first to eighth-first embodiments, in which the inorganic acid is one or more species selected from the group consisting of carbonic acid, hydrochloric acid, nitric acid, and sulfuric acid.

A ninth embodiment may provide the composition of any one of the first to eighth embodiments, in which the composition is a skin external application composition.

A tenth embodiment may provide the composition of any one of the first to ninth embodiments, in which the composition is for moisturizing the skin.

An eleventh embodiment may provide the composition of any one of the first to tenth embodiments, in which the composition is for preventing loss of skin elasticity or improving skin elasticity.

A twelfth embodiment may provide the composition of any one of the first to eleventh embodiments, in which the composition is a cosmetic composition.

A thirteenth embodiment may provide the composition of any one of the first to twelfth embodiments, in which the composition is a food composition.

A fourteenth embodiment may provide the composition of any one of the first to thirteenth embodiments, in which the composition is a pharmaceutical composition.

A fifteenth embodiment may provide a method of manufacturing the composition of any one of the first to fourteenth embodiments, the method comprises: micronizing a high molecular weight hyaluronic acid or salt thereof by adding an acidic pH adjuster to a solution that comprises the high molecular weight hyaluronic acid or salt thereof, in which the micronized high molecular weight hyaluronic acid or salt particle thereof has an average particle diameter of 100 nm or less, and a pH of the composition is 3.0 or more but less than 5.0.

A sixteenth embodiment may provide the method of the fifteenth embodiment, in which the micronizing of the high molecular weight hyaluronic acid or salt thereof further comprise adding a metal ion sequestering agent to the solution.

The seventeenth embodiment may provide the composition of any one of the first to fourteenth embodiments, in which the composition is a carrier composition for the transdermal delivery of an active substance, and the composition releases the active substance at a pH of 5 to 7.

The eighteenth embodiment may provide a skin external application composition comprising: an active substance; and a carrier for the transdermal delivery of the active substance, comprising the composition according to any one of the first to fourteenth embodiments.

The nineteenth embodiment may provide a skin external application composition of the eighteenth embodiment, in which the skin external application composition comprises the active substance in an amount of 0.01 wt % to 40 wt % based on the total weight of the skin external application composition.

The twentieth embodiment may provide the skin external application composition of the eighteenth or nineteenth embodiment, in which the active substance is physically captured in the carrier composition.

The twenty first embodiment may provide the skin external application composition of the eighteenth to twentieth embodiments, in which the skin external application composition is for moisturizing skin.

The twenty second embodiment may provide the skin external application composition of the eighteenth to twenty first embodiments, in which the skin external application composition is for preventing skin elasticity reduction or improving elasticity.

The twenty third embodiment may provide the skin external application composition of the eighteenth to twenty second embodiments, in which the skin external application composition is for whitening skin.

The twenty fourth embodiment may provide the skin external application composition of the eighteenth to twenty third embodiments, in which the skin external application composition is a cosmetic composition.

The twenty fifth embodiment may provide the skin external application composition of the eighteenth to twenty fourth embodiments, in which the skin external application composition is a pharmaceutical composition.

1.-17. (canceled)

18. A skin external application composition, comprising:
an active substance; and
a carrier for transdermal delivery of the active substance, comprising a high molecular weight hyaluronic acid or salt particles thereof; and an acidic pH adjuster,
wherein the acidic pH adjuster is an organic acid or inorganic acid,
wherein the high molecular weight hyaluronic acid or salt particles thereof have an average particle diameter of 100 nm or less,
wherein a pH of the composition is 3.0 or more but less than 5.0,
wherein the carrier captures the active substance, and
wherein the carrier releases the active substance at a pH of 5 to 7.

19. The skin external application composition of claim 18, wherein the skin external application composition comprises the active substance in an amount of 0.01 wt % to 40 wt % based on a total weight of the skin external application composition.

20. The skin external application composition of claim 18, wherein the active substance is physically captured in the carrier composition.

21.-23. (canceled)

24. The skin external application composition of claim 18, wherein the skin external application composition is a cosmetic composition.

25. The skin external application composition of claim 18, wherein the skin external application composition is a pharmaceutical composition.

26. The skin external application composition of claim 18, further comprising:
a metal ion sequestering agent.

27. The skin external application composition of claim 26, wherein the metal ion sequestering agent is one or more species of an alkaline salt of ethylene-diamine-tetraacetic acid (EDTA) and an alkaline salt of a phosphate-based compound.

28. The skin external application composition of claim 18, wherein a weight average molecular weight of the high molecular weight hyaluronic acid or salt thereof is 5 to 10,000 kDa.

29. The skin external application composition of claim 18, wherein a weight ratio of the high molecular weight hyaluronic acid or salt thereof to the acidic pH adjuster is 1:0.01 to 10.

30. The skin external application composition of claim 18, wherein the high molecular weight hyaluronic acid or salt thereof is comprised at 0.1 to 10 wt % based on a total weight of the composition.

31. The skin external application composition of claim 18, wherein the acidic pH adjuster is comprised at 0.01 to 50 wt % based on a total weight of the composition.

32. The skin external application composition of claim 18, wherein the organic acid is one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, and gluconolactone, and
wherein the inorganic acid is one or more species selected from the group consisting of carbonic acid, hydrochloric acid, nitric acid, and sulfuric acid.

33. A method for moisturizing skin, preventing skin elasticity reduction, improving skin elasticity or whitening skin, comprising: administering an effective amount of the composition according to claim 18 to skin of a subject in need thereof.

34. The method of claim 33, wherein the composition further comprises a metal ion sequestering agent.

35. The method of claim 34, wherein the metal ion sequestering agent is one or more species of an alkaline salt of ethylene-diamine-tetraacetic acid (EDTA) and an alkaline salt of a phosphate-based compound.

36. The method of claim 33, wherein a weight average molecular weight of the high molecular weight hyaluronic acid or salt thereof is 5 to 10,000 kDa.

37. The method of claim 33, wherein a weight ratio of the high molecular weight hyaluronic acid or salt thereof to the acidic pH adjuster is 1:0.01 to 10.

38. The method of claim 33, wherein the high molecular weight hyaluronic acid or salt thereof is comprised at 0.1 to 10 wt % based on a total weight of the composition.

39. The method of claim 33, wherein the acidic pH adjuster is comprised at 0.01 to 50 wt % based on a total weight of the composition.

40. The method of claim 33, wherein the organic acid is one or more species selected from the group consisting of citric acid, phosphoric acid, fumaric acid, ascorbic acid, ferulic acid, succinic acid, lactic acid, hyaluronic acid, malic acid, butyric acid, formic acid, acetic acid, salicylic acid, glycolic acid, tartaric acid, and gluconolactone, and
wherein the inorganic acid is one or more species selected from the group consisting of carbonic acid, hydrochloric acid, nitric acid, and sulfuric acid.
